(19) United States
(12) Patent Application Publication
(10) Pub. No.: US 2023/0166076 A1
(43) Pub. Date: Jun. 1, 2023

(54) INVASIVE TOOL WITH CONTROLLED RIGIDITY AND METHODS OF CONSTRUCTION

(71) Applicant: ENDOWAYS LTD., Caesarea (IL)

(72) Inventor: Noam Shaul SHAMAY, Moshav Elyakhin (IL)

(73) Assignee: ENDOWAYS LTD., Caesarea (IL)

(21) Appl. No.: 18/102,512

(22) Filed: Jan. 27, 2023

Related U.S. Application Data
(63) Continuation of application No. PCT/IL2021/050920, filed on Jul. 29, 2021.
(60) Provisional application No. 63/059,175, filed on Jul. 31, 2020.

(51) Int. Cl. A61M 25/00

(52) U.S. Cl. CPC A61M 25/0053; A61M 25/0012

(57) ABSTRACT

An invasive tool with controlled rigidity has a distal portion which supports a controlled rigidity profiled structure to provide a desired profile of rigidity. In one embodiment, the profiled structure is formed by helical winding of a strip of side by side disposed microwires into a sequence of same distally extending sections having the same rigidity. For control of rigidity, selected microwires from chosen sections are terminated. Thereby, the degree of relative rigidity each one of the chosen sections may be reduced to provide a profile of desired rigidity. Construction of the profiled structure uses a wire winding machine, and a laser machine to terminate microwires. Thereafter, the profiled structure is integrated in the distal portion of an invasive tool, as well known in the art, whereby an invasive tool with controlled rigidity is provided.

### CROSS-REFERENCE TO RELATED APPLICATIONS

This application is a Continuation of International Application No. PCT/IL2021/050920, filed Jul. 29, 2021, which claims the priority of U.S. Provisional Patent Application No. 63/059,175, filed Jul. 31, 2020, the entire contends of both of which are incorporated herein by reference.

### TECHNICAL FIELD

The embodiments described hereinbelow relate to invasive tools such as catheter devices for in vivo use, and in particular, to invasive tools for interventions which require a longitudinal extension with various levels of controlled rigidity.

### SUMMARY OF INVENTION

The embodiments described hereinbelow refer to an invasive tool having a distal portion with a profile of controlled degree of rigidity. Controlled rigidity means that segments and sections of the distal portions of the invasive tool may be designed to have a longitudinally extending varied and varying degree rigidity in bending rigidity and/or in torque rigidity.

To achieve an invasive tool having a distal portion with a controlled degree of rigidity, use is made of a profiled structure PRFSTR which is processed to have and to impart the desired quality of controlled rigidity to the invasive tool. This means that when a profiled structure PRFSTR is embedded, or integrated in the distal portion of an invasive tool, that same invasive tool is provided with a controlled degree of rigidity.

The profiled structure PRFSTR is made from an initial number of microwires which are acquired as a flat strip of side by side disposed microwires, and are packaged in adherence to a substrate for ease of handling. Assuming a strip having an initial number of microwires of the same length, that is wound helically in longitudinal extension, then each turn of the strip forms a section with the same initial number of microwires, whereby each section has the same rigidity. To provide that helical winding with a controlled rigidity, thus sections with a different but controlled degree of rigidity, selected microwires MWR are terminated and removed from chosen sections SEC. Evidently, for identical microwires, sections SEC with less microwires are less rigid than sections with more microwires.

Termination of selected microwires, thus deletion and removal of the distal portion of a selected microwire, may be achieved by either termination of the selected microwire before helical winding of the strip, or termination after helical winding of the strip.

As one alternative, a strip of microwires may be helically wound into a tubular structure, and thereafter, be profiled, i.e. selected microwires from chosen sections of the tubular structure may be terminated. Thereby, the termination may create sequences having a different number of microwires, thus a variety of degrees of rigidity, whereby the tubular structure may be sculptured into a profiled structure having a controlled rigidity.

As another alternative, selected microwires may first be terminated while still on the strip, to become a processed strip, or cut strip, and only thereafter may the cut strip be wound, which winding provides the profiled structure having a controlled rigidity.

Evidently, the termination of a selected microwire from a chosen section reduces the degree of rigidity thereof. It may be said that the termination of one microwire reduces the degree of rigidity of that section by one degree of rigidity. Moreover, the termination of a selected microwire from one section propagates to therefrom distally extending sections, which causes a same reduction of rigidity to those distally extending sections.

The degree of rigidity of a section may be indicated as the number of coils of microwires a section supports. Most often, the most proximal section of the profiled structure has the highest degree of rigidity, thus the degree of rigidity which is equal to the numerical value of the maximal number of microwires on a strip, which is usually the initial number of microwires in that strip. The minimal degree of rigidity is evidently equal to at least the most distal section of the profiled structure. One may say that to be regarded as a profiled structure, at least one microwire has to be terminated from at least one section which is distal from the first most proximal section, when that one section supports the maximal number of microwires.

The invasive tool with controlled rigidity may be constructed by providing a strip supporting an initial number of microwires. As a first step, a wire winding machine may be used for winding the strip into consecutive sections of microwires for forming a tubular structure. Then, as a second step, a laser beam machine may be provided and operated on the tubular structure for terminating selected microwires from chosen sections, for forming a profiled structure having sections of predetermined degree of rigidity. Finally, the profiled structure may be integrated in the distal portion of the invasive tool, whereby controlled rigidity thereof is achieved.

A laser beam from a laser beam machine, or laser machine, may be operated for terminating a microwire MWR and for forming a microwire terminated end MWRND, and the same laser machine may be further operated for welding the terminated end MWRND by a weld point WLDPNT, to an adjacent microwire MWR, when present.

The profiled structure is a simple machine part made from wound microwires which are terminated. By being of such simple design and construction, the profiled structure is adapted for design and simulation of operation by use of respectively, computer aided design programs and simulation programs running on a computing processor. In addition, by requiring a winding and a termination process for construction, the profiled structure is adapted for construction by operation of computer aided manufacturing programs 300 controlling a wire winding machine and a laser beam machine commanded by a computing processor.

As an alternative method, the invasive tool with controlled rigidity may be constructed still by providing a strip supporting an initial number of microwires, but differently, by a first step using a laser beam machine for terminating selected microwires MWR from the strip, and thereby forming a profiled strip, or cut strip. Then, by a second step, a wire winding machine may be used for winding the profiled strip into a profiled structure of consecutive sections, which have a profiled predetermined degree of rigidity. The alternative method thus first terminates selected microwires from the strip, and then winds the strip into a profiled structure. This is contrary to first winding the strip and thereafter, terminating selected microwires MWR.

### Technical Problem

Typically, common available invasive medical implements IMPL have a distal portion which in practice, proves sometimes to be either too rigid, or too flexible, or lack reliable torque compliance. Even though those common invasive medical implements IMPL may be provided with some specific flexibility, the lack of rotational rigidity, or torque compliance, is a major deficiency. Common invasive medical implements IMPL may include catheters, microcatheters, guidewires, endoscopes, cardial leads, duodenoscopes, enteroscopes, stent retrievers, occlusion crossing devices, and more.

It would therefore be advantageous to provide an invasive tool which has longitudinally extending degrees profiled rigidity configured to meet the needs and desires of a practitioner, and which may be designed for example, for a specific intervention, to permit travel through the twists and turns of the meanders of the vasculature. Even if not tailor-made for a specific intervention, an off the shelf collection of invasive tools to choose from, and having a profile with different sections of controlled degrees of rigidity, including better controlled longitudinal rigidity, and torque rigidity, i.e. rotational rigidity, or torque transmission compliance, would be of benefit.

FIG. 1 depicts an example of a partial cross-section of a portion of the distal portion DSTPRT of a commonly available invasive medical implements IMPL, such as a catheter CAT, having an interior tubular passage INT. Typically, a catheter CAT has a rigidity or stiffness which is dictated by concentric layers of material, such as for example, a tubular liner LINR, a stranded tube STRTUB, and filler FILR. First, a tubular liner LINR which forms a channel with an axis X, and may be made as a tube of artificial material. Second, a stranded tube STRTUB which covers the liner LINR. Third, a filler FILR which as a thin film of polymer which coats the stranded tube STRTUB, to form a smooth exterior surface on the distal portion DSTPRT.

The common practice to reduce the rigidity of the distal portion DSTPRT, is to skive the filler FILR, which practice is neither practical nor especially effective, in particular since skiving is a side cut, that affects the angular rigidity and thus the rotational stiffness, or torque compliance response. Furthermore, skiving causes the distal portion DSTPRT to remain curved, which may cause difficulties of navigation in vessels.

Other methods for dealing with rigidity, different from the one depicted in FIG. 1, are available, such as catheters CAT having an element produced by centerless grinding, to provide a distal decrease of rigidity. Like still more commonly available invasive tools, these tools do not respond to needs as desired, and are expensive by requiring intricate processes of manufacture. Therefore, an invasive tool well suited for the task and having a desired controlled rigidity made to achieve a profile of desired degree of rigidity is needed. This means that such an invasive tool should provide controlled degrees of rigidity, i.e. in bending moment and in torque compliance.

### Solution to Problem

The solution includes an invasive tool having a distal portion which supports a distribution of sections wound from microwires, wherein the degree of rigidity of the sections is controllable by selective termination of microwires. Thereby, a selected predetermined profile of sections having distally extending varying or constant degrees of rigidity is provided to achieve an invasive tool with controlled rigidity.

FIG. 2 illustrates an example of a segment SGMNT of a hollow tubular structure TUBSTR which extends from a proximal PRX to a distal DST direction. FIG. 2 shows a pair of parallelly disposed microwires MWR, marked a and b, which are wound helically into coils CL and form successive sections SEC of pairs of coils. The microwires MWR a and b may be wound either in a clockwise CW direction of right-hand turns, or in an a counterclockwise CCW direction of winding in left hand turns. The two most proximal PRX sections SEC, marked SEC1, and SEC2 are formed by pairs of wound microwires MWR a and b. Each one section SEC, here SEC1 and SEC2, has a same number of microwires MWR, a and b, and has a same degree of rigidity, designated as DOR1. Consecutive sections SEC having a same number of coils CL of microwires MWR, form a stack STK, such that the two sections SEC1 and SEC2 form a first proximal PRX stack STK1.

The proximal PRX stack STK1 is followed by three same sections, SEC3, SEC4, and SEC5, which form a second stack STK2. In the third section SEC3, in the second stack STK2, the first microwire a is missing by having been terminated at the end of a full turn in section SEC2, thus cut away and removed. Thereby, the three distal sections SEC3, SEC4, and SEC5 have just one microwire b, and therefore, have a lesser second degree of rigidity DOR2, which is lower than the degree of rigidity DOR1 of the first section SEC1 and the second section SEC2, both last having two microwires MWR, a and b.

A reduction of rigidity of a selected section SEC of the structure TUBSTR requires the processing of that selected section SEC. As shown in FIG. 2, this may be achieved by the termination of a microwire MWR from a section SEC. The removal of a microwire MWR from a proximal PRX section SEC reduces the degree of rigidity not only of that proximal section SEC, but also reduces the degree of rigidity of the sections SEC extending distally away therefrom. With a tubular structure TUB STR, the most proximal PRX section SEC supports an initial number of INNBR of microwires MWR. After the reduction of rigidity, thus termination of microwire(s) MWR, the most distal DST section SEC supports at least one microwire MWR. and at most the initial number INNBR of wires MWR minus one.

It is noted that in FIG. 2 the two proximal sections SEC1 and SEC2 are tightly wound but this is not necessarily so, since and interstice INTRST may separate between the microwires MWR, which occurs mostly in the most distal segments SEG.

There has thus been described a rigidity control process able to control the rigidity of portions of a tubular structure TUBSTR wound helically from microwires MWR formed as sections SEC and stacks STK. The same rigidity control process is applicable to invasive tools and especially so for control of the rigidity of the distal portion thereof, which permits to design invasive tools having a predetermined profile of degrees of rigidity.

### Advantageous Effects of Invention

The embodiments described herewith provide a simple solution for the provision of invasive tools 100 having a distal portion DSTPRT with a controlled profile of rigidity. If desired, the profile of rigidity may easily be configured to match the requirements of a specific medical intervention. Use is made of well-known methods of design, and manufacturing and take advantage of standard processes and machinery, which include wire winding and wire termination and removal. Furthermore, the construction of a controlled invasive tool according to the proposed solution is adapted for processor controlled design and manufacture, by use of computer aided design and computer aided manufacturing processes.

### BRIEF DESCRIPTION OF DRAWINGS

Non-limiting embodiments of the invention will be described with reference to the following description of exemplary embodiments, in conjunction with the figures. The figures are generally not shown to scale and any measurements are only meant to be exemplary and not necessarily limiting. In the figures, identical structures, elements, or parts that appear in more than one figure are preferably labeled with a same or similar number in all the figures in which they appear, in which:

FIG. 1 depicts a partial cross-section of portion of a distal portion of a commonly available invasive medical implement IMPL,

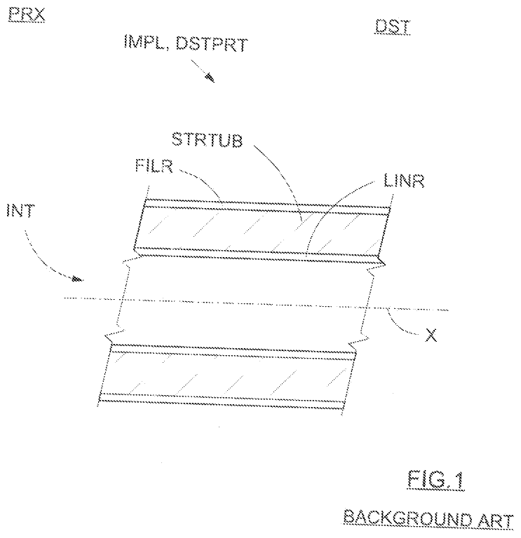

FIG. 2 illustrates an example of a segment of a wound hollow tubular structure TUB STR wherefrom a microwire MWR has been terminated,

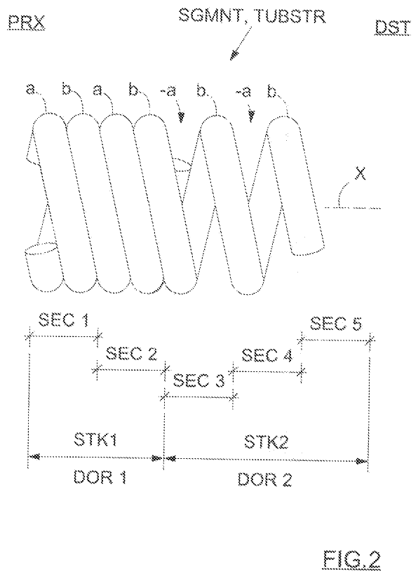

FIG. 3A illustrates a segment SGMNT of a tubular structure TUBSTR having a uniform degree of rigidity,

FIG. 3B, depicts a profiled structure PRFSTR with a decreasing degrees of rigidity DOR,

FIG. 3C depicts a profiled structure PRFSTR wound from a strip STRP of microwires MWR which are separated apart by interstices INTRST,

FIGS. 4A to 4C show strips STRP of microwires MWR,

FIG. 5 illustrates a segment SGMNT of a concentric profiled assembly CNCPRF with concentric disposed profiled structures PRFSTR,

FIG. 6 depicts a distal DSTL portion of a controlled rigidity invasive tool 100,

FIG. 7 is a block diagram of a design and manufacturing process,

FIGS. 8-9 are block diagrams of a manufacturing processes, and

FIG. 10 schematically illustrates a controlled rigidity invasive tool 100.

### DESCRIPTION OF EMBODIMENTS

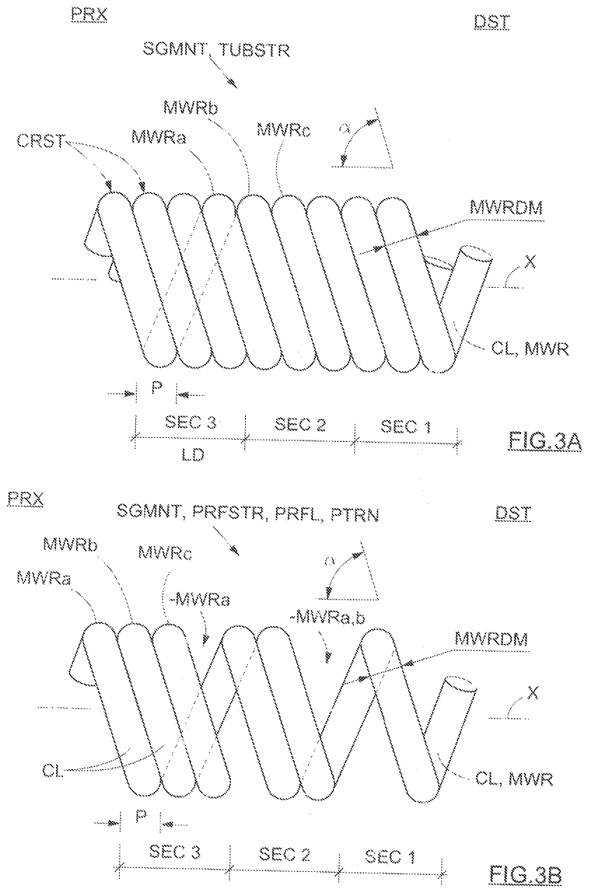

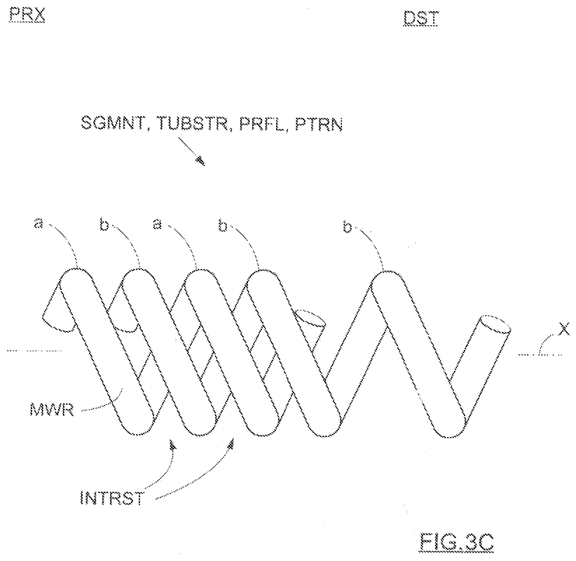

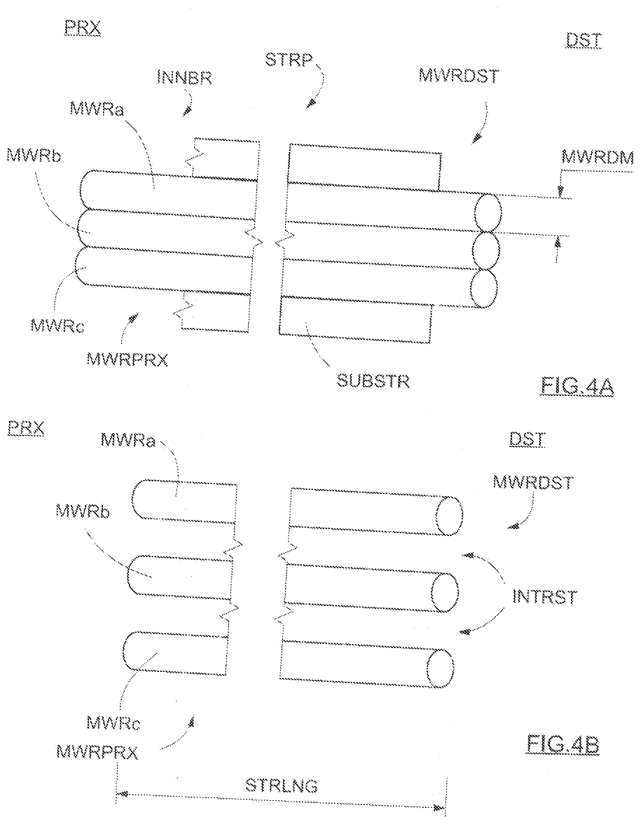

FIG. 3A illustrates an exemplary embodiment of a segment SGMNT of a distally extending portion of a hollow longitudinal tubular structure TUBSTR of uniform degree of rigidity, that after further processing, may be used to provide the distal portion DST of an invasive tool 100 with controlled rigidity. To achieve a tubular structure TUBSTR having a finely controlled rigidity, or degree of stiffness, microwires MWR may be used. Microwires MWR may have an exterior diameter MWRDM which is measured in microns, also known as micrometers, and are thus very thin when compared to wires of millimetric size. For example, a microwire MWR may have an exterior diameter MWRDM of 15 microns, or 0.015 mm. With the exemplary embodiments described herewith, a tubular structure TUBSTR may be wound from a plurality of microwires MWR packaged as a flat strip STRP of microwires MWR.

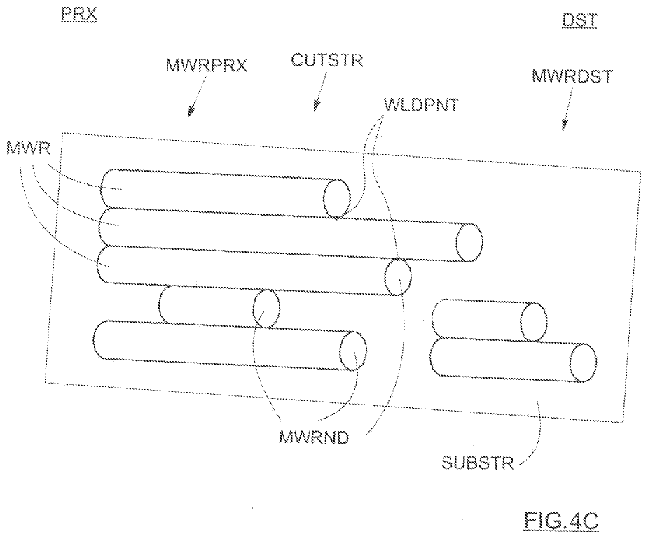

In FIG. 4A, a flat pack, or flat strip STRP of microwires MWR is shown to be similar to a flat flexible cable, or FFC, used in flexible electronics, reference to which is found in the Internet at Wikipedia under “Flat flexible cable”, or FFC. Like a flat flexible cable FFC, a strip STRP is flat and flexible, and may be packaged on a flat and flexible substrate SUBSTR or support, which may be easily removed before use if necessary. In the exemplary embodiments described and depicted herewith, for the sake of ease of illustration, microwires MWR are referred to and depicted as being of round, thus circular cross-section, but other cross-sectional shapes may also be used if desired. Furthermore, the microwires MWR in a flat strip STRP may be ordered in a collaterally disposed distribution, tightly together in mutual collateral side-by-side contact, or be distanced apart from each other by an interstice INTRST, as shown in FIG. 4B. A flat strip STRP may hold an initial number INNBR of microwires MWR, ranging from two, to ten, twenty, thirty, and even more microwires MWR, according to need and desired selection. The microwires MWR are shown to be packaged on a substrate SBSTR.

FIG. 4A depicts a flat strip STRP of microwires MWR, with an initial number INNBR of three microwires MWR arranged laterally in side-by-side contact, marked as MWRa, MWRb, and MWRc. The initial number INNBR number of microwires MWR may range from 1 to m, where m is a positive integer number.

FIG. 4B depicts a flat strip STRP of length STRLNG having an initial number INNBR of microwires MWR which are separated apart by interstice INTRST, contrary to the closely wound microwires MWR shown in FIG. 4A. Microwires MWR may thus be wound in tight compression and in separation apart by interstices INTRST. The distance of separation apart between successive coils CL of microwires MWR, thus the dimension of an interstice INTRST, are an additional technique for the control of the degree of rigidity of a tubular structure TUBSTR and of a profiled structure PRFSTR.

FIG. 4C illustrates a flat strip STRP showing terminated microwires MWR supported on a substrate SUBSTR. As described hereinbelow, microwires MWR may be terminated from a strip STRP before being wound. When terminated, a microwire MWR has a microwire terminated end TRMND, which when cut by a laser beam, may be weld to a thereto adjacent microwire MWR at a weld point WLDPNT, if such an adjacent microwire is present. It is noted that microwires MWR may be terminated not only at their distal side MCWDST or at their proximal side MWRPRX, but anywhere along the strip STRP.

FIG. 3A thus depicts a segment SGMNT of a longitudinally extending tubular structure TUBSTR which is wound tightly and helically from a flat strip STRP, holding an initial number INNBR of same microwires MWR, to form a sequence of three distally extending sections SEC of coils CL, or three complete turns of the strip STRP. The three sections SEC, marked from proximal PRX to distal DST direction as SEC3, SEC2, and SEC1, have the same section length, and are tightly wound and compressed together for the sake of rigidity. Each one turn of winding of the strip STRP forms a section SEC, and therefore, each one of the three sections SEC has a same initial number INNBR of microwires MWR, a same number of tightly wound coils CL of microwire MWR, and a same degree of rigidity DOR.

In FIG. 3A, the crests CRST of the wound microwires MWR of the tubular structure TUBSTR may be considered and compared for example, to the crests of a screwthread of a male mechanical fastener, such as a screw or a bolt. The sections SEC of the segment SGMNT support three wound microwires MWR, thus microwires MWRa, MWRb, and MWRc, the crests CRST of which are similar to the crests of a male screwthread with three starts, and thereby, when rotated, the segment SGMNT translates accordingly. When the segment SGMNT shown in FIG. 3A is rotated in a body vessel, these crests CRST may engage tissue of the vasculature and enhance translation therethrough. Still like mechanical fasteners, the microwires MWR may be helically wound with a pitch PT and a lead LD. The pitch PT is the distance separating apart between the crest CRST of two consecutive coils CL. For collateral tightly wound coils CL, the pitch PT equals the exterior diameter MWRDM of a microwires MWR. The lead LD is the axial translation distance covered for each one 360° turn of rotation of the tubular structure TUB STR, or of a profiled structure which is described hereinbelow. For a section SEC having three wound together microwires MWR, similar to a screwthread having three starts, the lead distance LD equals to three pitch distances PT, thus three times the exterior diameter MWRDM of a microwire MWR. However, for a screwthread with one start, the pitch distance PT equals the lead distance LD.

For the sake of torque rigidity, or rotational stiffness/rigidity, the helix angle α, shown in FIG. 3A, of winding of the microwires MWR forming the structure TUBSTR may be rather large when compared to the helix angle of a mechanical fastener, and may be selected in the range of 30° to 60°, and preferably as about 45°. Contrary to a small helix angle of 10 to 15 degrees as for mechanical single thread screws, a rather large helix angle α may provide an enhanced longitudinal rigidity as well as an enhanced rotational rigidity. Thereby, a proximally applied angular rotation imparted to a proximal portion PRXPRT of the tubular structure TUBSTR may be responded by the distal portion DSTPRT of the structure TUB STR by a monotonous continuous angular compliant rotation. This is especially so when rotated in the direction of winding, and is also true for a profiled structure PRFSTR. Such a behavior of rotational compliance with proximally PRX delivered input torque is different from common invasive medical implements IMPL having skived fillers FILR which deform into curved distal portions DSTPRT due to their asymmetric skived cut. Contrary to a controlled rigidity invasive tool 100, the distal portion DSTPRT of a skived catheter CAT remains curved. When rotated in a vessel of a body, the curved distal portion DSTPRT of a skived catheter CAT is detrimental to rotational compliance when proximally torqued.

To reduce the rigidity DOR of a section SEC, the number of coils CL of microwire MWR has to be reduced by at least one by a manufacturing process. That same process of rigidity control may be used to longitudinally vary the rigidity of the distal portion DSTPRT for a controlled rigidity invasive tool 100.

FIG. 3B illustrates an exemplary embodiment of a segment SGMNT of a profiled structure PRFSTR made from a tubular structure TUBSTR by termination of microwires MWR. The portion of tubular structure TUBSTR from which the profiled structure PRFSTR of FIG. 3B is made is shown in FIG. 3A, as a uniform winding of three sections SEC of uniform degree of rigidity.

To create a profiled structure PRFSTR with a distally DST decreasing rigidity DOR, advantage is taken from the tubular structure TUBSTR shown in FIG. 3A, having three sections SEC, indicated from proximal PRX to distal DST direction as SEC3, SEC2, and SEC1. The proximal PRX first section SEC3 supporting the initial number INNBR of microwires MWR is not processed, but from the second middle section SEC2, the first microwire MWRa has been terminated, as indicated by -WRa. This termination caused the removal and deletion of the distal portion MWRDST of the first microwire MWR which prior to termination, also propagated distally DST to the most distal DST section SEC1. Thereby, the second section SEC2 and the third section SEC1 are left with two microwires MWR, namely microwire MWRb and microwire MWRc. In turn, the second microwire MWRb is terminated, as indicated by -WRb. The result is that the third section SEC1 now supports only the third microwire MWRc whereby the degree of rigidity DOR of the sections SEC of the profiled structure PRFSTR diminishes smoothly and monotonously, in in distal DST direction, by one microwire MWR, or one count of rigidity. A count of rigidity, or degree of rigidity DOR may be proportional, or equal to the number of microwires MWR in a section SEC.

The tubular structure TUBSTR illustrated in FIG. 3A, has thus been processed into a profiled structure PRFSTR which is shown in FIG. 3B. The profiled structure PRFSTR presents a profile PRFL of distally descending or reduced degree of rigidity DOR stretching over three sections SEC. The same rigidity control process by termination of microwires MWR may evidently be applied to a plurality of more than three sections SEC supported by the length of the distal portion DSTPRT of a controlled rigidity invasive tool 100. For example, the distal portion DSTPRT of the invasive tool 100 may have a length of 10 cm to 30 cm, or more. The sections SEC of the distal portion DSTPRT of the invasive tool 100 may be wound from microwires MWR, provided on a strip STRP, having a microwire exterior diameter MWRDM ranging from 0.015 mm to 0.5 mm.

FIG. 3C shows an exemplary embodiment of a profiled structure PRFSTR wound from a strip STRP of microwires MWR which are separated apart by interstices INTRST, as shown in shown in FIG. 4B. Interstices INTRST may be formed on a strip STPR, or at least on a portion thereof, or be formed by termination of microwires MWR. It is understood that interstices INTRST reduce the degree of rigidity DOR of a section SEC when compared to a section SEC of same length wound from tightly compressed together coils CL of microwires MWR. The wider the interstice INTRST, the lower the degree of rigidity DOR. A strip STRP of microwires MWR, either disposed tightly together, or having microwires MWR separated apart by an interstices INTRST, may be wound either in a clockwise CW direction, or in a counterclockwise CCW direction.

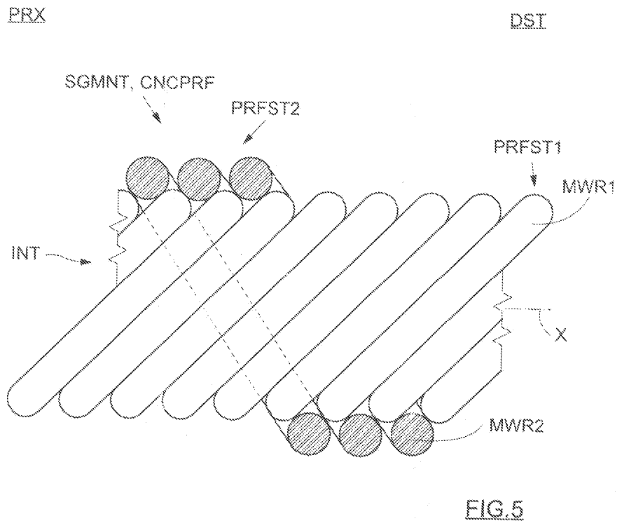

FIG. 5 illustrates an exemplary embodiment of a segment SGMNT of a concentric profiled assembly CNCPRF which includes concentrically disposed profiled structures PRFSTR, as may be used in the distal portion of a rigidity controlled invasive tool 100. A concentric profiled assembly CNCPRF, includes at least two concentrically disposed profiled structures PRFSTR. Although FIG. 5 depicts two profiled structure indicated as PRFST1 and PRFST2, the number of concentric profiled structures PRFSTR is not limited to two. Each one profiled structure PRFSTR, here PRFST1 and PRFST2, may have a same or a different one of: initial number INNBR of microwires MWR, microwire exterior diameter MWRDM, type of material, number of sections SEC, direction of winding, and helix angle α of winding.

The first profiled structure PRFST1 is shown to support a second profiled structure PRFST2 which is disposed concentrically thereover. Each one profiled structure, PRFST1, PRFST2, may have the same direction of winding, either clockwise CW or counterclockwise CCW.

If desired, more than two profiled structures PRFSTR may be disposed concentrically over the first tubular structures STRC1, even though not shown as such in FIG. 5. A plurality of concentrically disposed profiled structure PRFSTR provides an increased rigidity, thus an increase of both the deflection rigidity and the torque compliance response of the distal portion DSTPRT of the invasive tool 100.

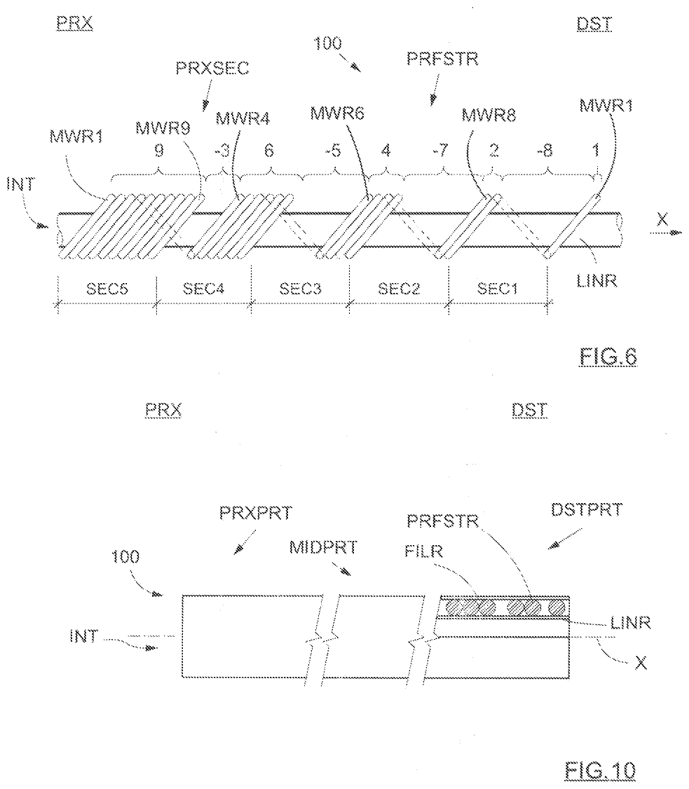

FIG. 6 depicts an exemplary embodiment of a profiled structure PRFSTR, with five sections SEC, ready for integration, to form the rigidizing portion of and in the distal portion DSTPRT of an invasive tool 100. The first most proximal section SEC5, or first proximal section PRXSEC, is wound from an initial number INNBR, here nine microwires MWR, and the four more therefrom distally extending sections SEC, extend from a fourth section SEC4 to a first section SEC1. In the exemplary embodiments described hereinabove, the termination of one microwire MWR per section SEC provided a rather smooth monotonous distal DSTL reduction of rigidity of the profiled structures PRFSTR. However, in FIG. 6, the first and most proximal PRX section SEC5 has nine microwires MWR and more than one microwire MWR is terminated in the therefrom distally DST extending sections SEC. Three microwires MWR have been terminated from the fourth section SEC4, two more microwires MWR have been terminated from the third section SEC3, and the second section SEC2, and thereby, the first section SEC1 is left with one microwire MWR. Hence, a profiled structure PRFSTR may have a controlled degree of rigidity which may be selected as one of, or a combination of: a constant rigidity, a decreasing rigidity, a smooth and/or an abrupt change of rigidity.

The rigidity control process of microwire MWR termination is thus applicable to produce a profiled structure PRFSTR with sections SEC having a constant degree of rigidity, and/or a decreasing degree of rigidity with smooth monotonous or abrupt changes of rigidity. Monotonous is regarded as being a change of degree of rigidity caused by the termination of a single microwire MWR.

Although not depicted herewith, and should that be desired or practical, the rigidity control process of microwire MWR termination may also be applied for providing a distally increasing degree of rigidity to portions of the profiled structure PRFSTR of a controlled rigidity invasive tool 100, or at sections SEC portions thereof.

A profiled structure PRFSTR made according to the controlled rigidity process of microwire MWR termination forms the rigidizing portion of and in the distal portion DSTPRT of a controlled rigidity invasive tool 100. Thereby, the invasive tool with the profiled structure PRFSTR mounted therein becomes an invasive tool 100 with controlled rigidity. Mounting of the profiled structure PRFSTR in the distal portion DSTPRT is common practice well known to those skilled in the art. For example, the interior passage INT of the profiled structure PRFSTR may be provided with a liner LINR, and the exterior portion thereof may be coated with a filler FILR.

### Construction

As described hereinabove, the process for providing controlled rigidity to the distal portion DSTPRT of an invasive tool 100 is based on the integration therein of a profiled structure PRFSTR having a predetermined rigidity profile PRFL. The profiled structure PRFSTR may be formed from a strip STRP of microwires which is wound into a tubular structure TUBSTR and thereafter processed, or else, by winding of an a priori processed strip STRP of microwires MWR into a profiled structure PRFSTR. Both the controlled tubular structure TUBSTR and the profiled structure PRFSTR are simple machine parts which may be constructed automatically by two processing machine steps.

The tubular structure TUBSTR may be first wound from a procured strip STRP of microwires MWR, and second, may be profiled into a desired rigidity profiled structure PRFSTR by controlled termination of microwires MWR. Else, microwires MWR from a strip STPR may first be terminated, and may thereafter be wound into the profiled structure PRFSTR. Termination of the microwires MWR may be achieved by mechanical cutting or by laser beam heat termination which has the advantage of welding, by a weld point WLD, of a microwire terminated end MWRND to a thereto in touch adjacent microwire MWR, when such one is present. The invasive tool 100 may be constructed with a tailor-made profile of rigidity dictated by a practitioner for a specific intervention, or as one item from a collection of invasive tools 100 having various rigidity profiles PRFL, made available as off the shelf items.

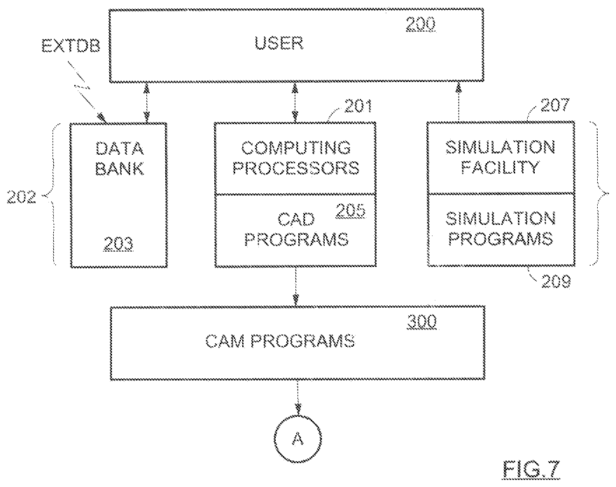

FIG. 7 illustrates a block diagram of an exemplary embodiment wherein computing processors 201 are provided to run a computer aided design facility 202 for the design and simulation of operation of a profiled structure PRFSTR.

A user 200, may be provided with the design facility 201, which is coupled to a data bank of information 203, and is further coupled in communication with other data banks EXTDB. The user 200 may next iterate the design of a desired product and simulate the operation thereof on, respectively, computer aided design programs 205, and computer aided simulation programs 207. Finally, a profiled structure PRFSTR may be produced by help of a computer aided manufacturing facility 301.

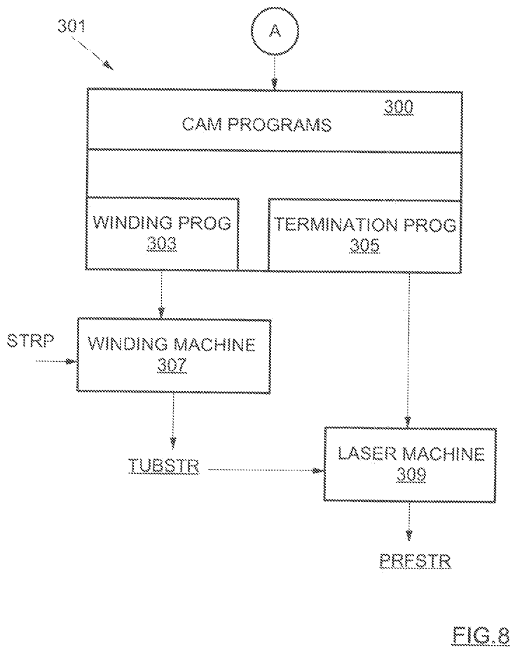

FIGS. 8 and 9 depict a computer aided manufacturing automatic facility 301, also operated by the computer aided design facility 202. However, even though operating the same machinery, each one of the FIGS. 8 and 9 offers a different production process sequence for the construction of the profiled structure PRFSTR for the distal portion of the invasive tool 100.

To produce the profiled structure PRFSTR, the computing processors 201 are configured to load the automatic computer aided manufacturing facilities 301 with the output of the design facility 201, for the operation of a microwire winding program 303 and of a microwire termination program 305. In turn, a wire winding machine, or winding machine 307, and a laser beam machine, or laser machine 309, are loaded with the respective programs of manufacture, namely 303 and 305. In both production processes, the microwires MWR on a strip STPR forms the raw material which is processed in a first step of production by one of the winding machine 307 and the laser machine 309.

In FIG. 8, as a first step of production, the wire winding machine 307 wraps the microwires MWR into a sequence of sections SEC to form a tubular structure TUB STR. Thereafter, in a second and last step of production, the tubular structure TUBSTR is processed by the laser machine 309 to terminate the selected microwires MWR and form a profiled structure PRFSTR, according to design. Thereby, the profiled structure PRFSTR is ready to form the rigidizing portion of and in the distal portion DSTPRT of a controlled rigidity invasive tool 100, by means well known to those skilled in the art.

In FIG. 9, as a first step of production, the laser machine 309 terminates the microwires MWR of the strip STPR according to design, whereby a cut strip CUTSTR of microwires MWR is obtained. Thereafter, in a second and last step of production, the wire winding machine 307 wraps the cut strip CUTSTR into the profiled structure PRFSTR. Thereby, the profiled structure PRFSTR is ready to form the rigidizing portion of and in the distal portion DSTPRT of a controlled rigidity invasive tool 100, by means well known to those skilled in the art.

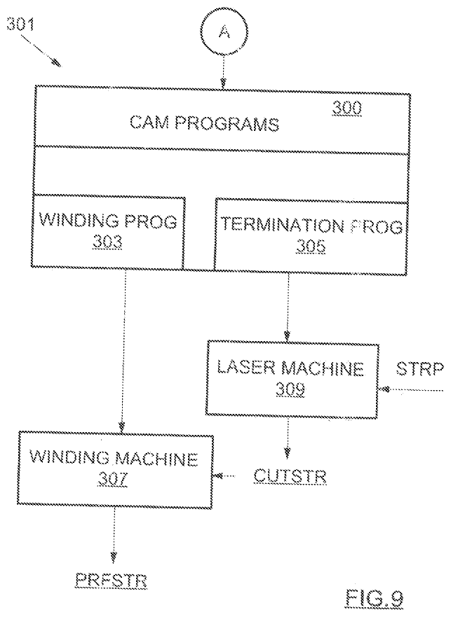

FIG. 10 illustrates an exemplary embodiment of a controlled rigidity invasive tool 100 with a partial cross-section of the distal portion DSTPRT thereof, a midportion MIDPRT, and a proximal portion PRXPRT. A liner LINR is shown in the tubular interior INT of the profiled structure PRFSTR which is covered by a filler FILR.

There has thus been described a profiled structure PRFSTR for the control of rigidity of an invasive tool PRFSTR and methods of construction thereof. The profiled structure PRFSTR is characterized by having an initial number INNBR of microwires MWR supported on a strip STRP, and by sections SEC of microwires MWR, wherein each one section SEC is wound by one turn of helical winding of the strip STRP, whereby the profiled structure PRFSTR is configured into controlled rigidity by termination of selected microwires MWR from chosen sections SEC.

### INDUSTRIAL APPLICABILITY

Invasive tools with controlled rigidity and methods of construction thereof are applicable in industries producing medical apparatus.

1. An invasive tool with controlled rigidity having a distal portion which supports a controlled rigidity profiled structure, the profiled structure comprising:
an initial number of microwires supported on a strip, and
sections of coils of microwires,
wherein each one section is wound by one turn of helical winding of the strip, and
wherein the profiled structure is configured into controlled rigidity by termination of selected microwires, wherein the termination is one of termination before helical winding and termination after helical winding.

2. The invasive tool of claim 1, wherein the strip is configured as an easily removable flat and flexible substrate.

3. The invasive tool of claim 1, wherein:
the strip of microwires is helically wound into a tubular structure, and
the profiled structure is configured into controlled rigidity by termination of selected microwires from chosen sections of the tubular structure.

4. The invasive tool of claim 1, wherein the profiled structure is further configured into controlled rigidity by:
termination of selected microwires from the strip before helical winding thereof, and
helical winding of the strip after termination of selected microwires.

5. The invasive tool of claim 1, wherein the profiled structure is further configured into controlled rigidity by termination of a selected microwire from a chosen section to reduce rigidity thereof and from sections extending distally away therefrom.

6. The invasive tool of claim 1, wherein the strip is wound at a helix angle α ranging between 30° to 60°.

7. The invasive tool of claim 1, wherein the strip is wound at a helix angle α of about 45°.

8. The invasive tool claim 1, wherein the strip is configured to support the microwires in collaterally disposed distribution.

9. The invasive tool of claim 1, wherein:
a first proximal section has a highest degree of rigidity which is designated as an integer equal in number to the initial, and
a last distal section has a lowest degree of rigidity which is designated as an integer equal in number to a number of the microwires supported by the last distal section.

10. The invasive tool of claim 1, wherein the profiled structure is further configured into a rigidity controlled profile by termination of at least one microwire from at least one section which is distal from a first most proximal section.

11. The invasive tool of claim 1, wherein the strip is wound is one of: a clockwise direction of winding, and a counterclockwise direction of winding.

12. The invasive tool of claim 1, wherein a concentric profiled assembly includes concentrically disposed profiled structures, wherein each one profiled structure is wound from microwires having an exterior diameter which is one of a same exterior diameter, and a different exterior diameter.

13. The invasive tool of claim 1, wherein a concentric profiled assembly includes concentrically disposed profiled structures which are wound is one of: a clockwise direction of winding, and a counterclockwise direction of winding.

14. The invasive tool of claim 1, wherein sections of coils of microwires which are ordered in collaterally disposed distribution, are wound in one of: tight compression and separation apart by interstices.

15. A method for constructing an invasive tool with controlled rigidity the method comprising:
providing a strip supporting a plurality of microwires,
using a winding machine and a laser machine to form the strip into a tubular structure, wherein the tubular structure has a predetermined rigidity profiled structure constructed by one of:
(i) integrating the profiled structure in a distal portion of the invasive tool to achieve controlled rigidity thereof, forming the strip into a tubular structure having a predetermined rigidity profiled structure by using the winding machine and the laser machine in one of:
(i)(a) using the winding machine for winding the strip, and operating the laser machine for termination of selected microwires, to form the profiled structure, and
(i)(b) using the laser machine for termination of selected microwires from the strip, to form a cut strip, and operating the winding machine for helical winding of the cut strip to form the profiled structure, and
(ii) integrating the profiled structure in a distal portion of the invasive tool, wherein longitudinal rigidity and torque rigidity for torque transmission compliance is achieved.

16. The method of claim 15, wherein the laser machine operates a laser beam for terminating a microwire and for forming a microwire terminated end.

17. The method of claim 15, wherein the laser machine operates a laser beam for terminating a microwire, for forming a microwire terminated end, and for welding the terminated end by a weld point to a thereto adjacent microwire.

18. The method of claim 15, wherein sections have a degree of rigidity which is proportional to a number of microwires supported thereby.

19. The method of claim 15, wherein the profiled structure is adapted for design and simulation by operation of respectively, computer aided design programs and simulation programs running on a computing processor.

20. The method of claim 15, wherein the profiled structure is constructed for construction by operation of computer aided manufacturing programs running on a computing processor.
